(19) United States
(12) Patent Application Publication
(10) Pub. No.: US 2022/0146504 A1
(43) Pub. Date: May 12, 2022

(54) SYSTEM AND METHOD FOR AUTOMATED COLLECTION OF AEROSOL PARTICLES

(71) Applicant: THERMO ENVIRONMENTAL INSTRUMENTS LLC, Franklin, MA (US)

(72) Inventors: Jeffrey Socha, Boylston, MA (US); Arunava DUTTA, Winchester, MA (US); Robert BAILEY, Bellingham, MA (US)

(21) Appl. No.: 17/519,293

(22) Filed: Nov. 4, 2021

Related U.S. Application Data
(60) Provisional application No. 63/110,641, filed on Nov. 6, 2020.

(51) Int. Cl. G01N 33/543; B01L 3/00

(52) U.S. Cl. CPC G01N 33/54366; B01L 3/508; B01L 2300/14; B01L 2200/082; B01L 2300/0609; B01L 2200/025

(57) ABSTRACT

An embodiment of an assembly for isolating a substrate is described that comprises a vacuum source; a substrate; a receptacle configured to position the substrate and to operatively couple to the vacuum source; and a vessel configured to operatively coupled to the receptacle, wherein the substrate is configured to move from the receptacle to the vessel in response to a differential pressure applied by the vacuum source.

### CROSS-REFERENCE TO RELATED APPLICATIONS

The present application claims the priority benefit from U.S. patent application Ser. No. 63/110,641, filed Nov. 6, 2020. The disclosure of the foregoing application is incorporated herein by reference.

### FIELD OF THE INVENTION

The present invention is generally directed to a system configured to collect and capture aerosolized particles from a gas.

### BACKGROUND

It is generally appreciated that systems exist for the collection of aerosolized particles from air, examples of which are described in U.S. Pat. Nos. 6,435,043; 6,769,316; 6,867,413; and 6,898,990, each of which is hereby incorporated by reference herein in its entirety for all purposes. In general, the systems collect the aerosolized particles onto substrate material that then must be manually handled to remove for subsequent particle analysis.

It is also appreciated that some particles, particularly some types of biological material such as viral particles, may pose a health risk to individuals that come into contact with them. Further, manual contact with the substrate may add a source of contamination that will affect results intended to reflect to content of the particles in the sampled gas.

Therefore, a need exists for an automated solution to remove and isolate the substrate material for particle analysis without the risk to the health of individuals as well as to sample integrity.

### SUMMARY

Systems, methods, and products to address these and other needs are described herein with respect to illustrative, non-limiting, implementations. Various alternatives, modifications and equivalents are possible.

An embodiment of an assembly for isolating a substrate is described that comprises a vacuum source; a substrate; a receptacle configured to position the substrate and to operatively couple to the vacuum source; and a vessel configured to operatively coupled to the receptacle, wherein the substrate is configured to move from the receptacle to the vessel in response to a differential pressure applied by the vacuum source.

In some cases, the substrate is constructed of polyurethane foam, and may include a perimeter area configured to reduce stiction. The substrate may also be configured to capture and retain a plurality of particles that can include biological material such as viral particles.

Also, in some implementations the receptacle comprises a chamber that positions the substrate and may be in fluid communication with the vessel and the vacuum source. In certain embodiments the differential pressure is greater than about 50 mbar.

An embodiment of a method for isolating a substrate is also described that comprises positioning a substrate in a receptacle; and moving the substrate from the receptacle to the vessel in response to a differential pressure.

The above embodiments and implementations are not necessarily inclusive or exclusive of each other and may be combined in any manner that is non-conflicting and otherwise possible, whether they are presented in association with a same, or a different, embodiment or implementation. The description of one embodiment or implementation is not intended to be limiting with respect to other embodiments and/or implementations. Also, any one or more function, step, operation, or technique described elsewhere in this specification may, in alternative implementations, be combined with any one or more function, step, operation, or technique described in the summary. Thus, the above embodiment and implementations are illustrative rather than limiting.

### BRIEF DESCRIPTION OF THE DRAWINGS

The above and further features will be more clearly appreciated from the following detailed description when taken in conjunction with the accompanying drawings. In the drawings, like reference numerals indicate like structures, elements, or method steps and the leftmost digit of a reference numeral indicates the number of the figure in which the references element first appears (for example, element 110 appears first in FIG. 1). All of these conventions, however, are intended to be typical or illustrative, rather than limiting.

FIG. 1 is a functional block diagram of one embodiment of an aerosol collector instrument with an impactor;

FIG. 2 is a simplified graphical representation of one embodiment of the impactor of FIG. 1 with a substrate positioned in a receptacle;

FIG. 3 is a functional block diagram of one embodiment of the receptacle of FIG. 2 with a vessel and a vacuum source;

FIG. 4 is a functional block diagram of one embodiment of the receptacle and vessel of FIG. 3;

FIG. 5A is simplified graphical representation of one embodiment of the receptacle of FIGS. 3 and 4 with a chamber;

FIG. 5B is simplified graphical representation of one embodiment of the receptacle of FIGS. 3, 4, and 5A with a suction interface and an aperture; and

FIG. 5C is simplified graphical representation of one embodiment of the receptacle of FIGS. 3, 4, 5A, and 5B with a vessel interface as well as a protrusion and an aperture.

Like reference numerals refer to corresponding parts throughout the several views of the drawings.

### DETAILED DESCRIPTION OF EMBODIMENTS

As will be described in greater detail below, embodiments of the described invention include a system configured to automatically and safely collect and capture aerosolized particles from a gas. More specially the particles may include biological material such as viral particles or bacterial particles, and the gas may include ambient air, breath from a living organism, or other gas that may include aerosolized biological material.

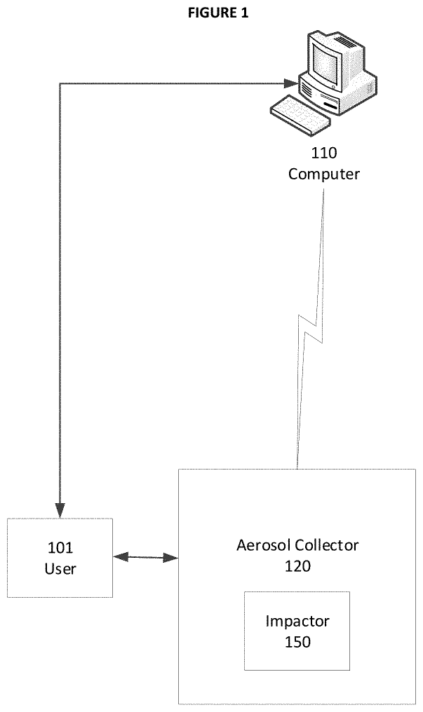

FIG. 1 provides a simplified illustrative example of user 101 capable of interacting with computer 110 and aerosol collector 120 with impactor 150. Embodiments of aerosol collector 120 may include any commercially available instruments configured for collecting particles from a gas. For example, aerosol collector 120 may include the ASAP 2800 instrument or the AerosolSense instrument available from Thermo Fisher Scientific. FIG. 1 also illustrates a network connection between computer 110 and aerosol collector 120, however it will be appreciated that FIG. 1 is intended to be exemplary and additional or fewer network connections may be included. Further, the network connection between the elements may include “direct” wired or wireless data transmission (e.g. as represented by the lightning bolt) as well as “indirect” communication via other devices (e.g. switches, routers, controllers, computers, etc.) and therefore the example of FIG. 1 should not be considered as limiting.

Computer 110 may include any type of computing platform such as a workstation, a personal computer, a tablet, a “smart phone”, one or more servers, compute cluster (local or remote), or any other present or future computer or cluster of computers. Computers typically include known components such as one or more processors, an operating system, system memory, memory storage devices, input-output controllers, input-output devices, and display devices. It will also be appreciated that more than one implementation of computer 110 may be used to carry out various operations in different embodiments, and thus the representation of computer 110 in FIG. 1 should not be considered as limiting.

In some embodiments, computer 110 may employ a computer program product comprising a computer usable medium having control logic (e.g. computer software program, including program code) stored therein. The control logic, when executed by a processor, causes the processor to perform some or all of the functions described herein. In other embodiments, some functions are implemented primarily in hardware using, for example, a hardware state machine. Implementation of the hardware state machine so as to perform the functions described herein will be apparent to those skilled in the relevant arts. Also in the same or other embodiments, computer 110 may employ an internet client that may include specialized software applications enabled to access remote information via a network. A network may include one or more of the many types of networks well known to those of ordinary skill in the art. For example, a network may include a local or wide area network that may employ what is commonly referred to as a TCP/IP protocol suite to communicate. A network may include a worldwide system of interconnected computer networks that is commonly referred to as the internet, or could also include various intranet architectures. Those of ordinary skill in the related art will also appreciate that some users in networked environments may prefer to employ what are generally referred to as “firewalls” (also sometimes referred to as Packet Filters, or Border Protection Devices) to control information traffic to and from hardware and/or software systems. For example, firewalls may comprise hardware or software elements or some combination thereof and are typically designed to enforce security policies put in place by users, such as for instance network administrators, etc.

As described herein, embodiments of the described invention include an automated solution to remove and isolate substrate material from an instrument used to capture particles from a gas. Importantly, the solution substantially eliminates human contact with the substrate the preserve integrity of the collected sample and protect individuals from potentially harmful pathogens.

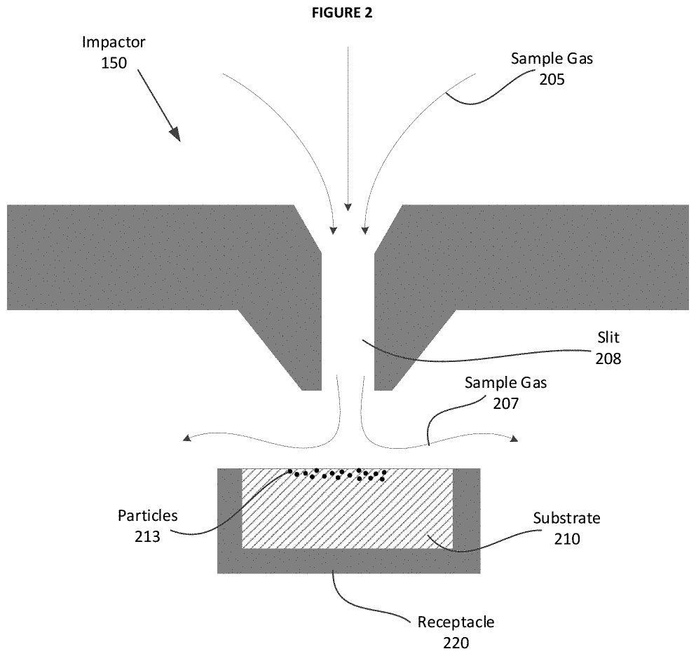

FIG. 2 provides a simplified illustrative example of impactor 150 that concentrates a flow of sample gas 205, typically containing particles 213 sampled from the ambient environment at a location, through slit 208 to impact substrate 210. Sample gas 205 impacts with substrate 210, depositing particles 213 onto the surface of and/or into the material of substrate 210, whereupon sample gas 207 exits, substantially without particles 213.

Substrate 210 may include a variety of materials configured to capture particles of interest and subsequently easily release the particles for analysis. Further, in some embodiments substrate 210 may include a substance or combination of substances configured to enhance capture and/or release of particles, stabilize biological particles, and/or enhance the viability of biological particles (e.g. the substance may be coated onto and/or impregnated into substrate 210). For example, substrate 210 may include polyurethane foam, porous polymers, glass or ceramic media, sintered material, electrically charged conductive media, or other substance known in the art. Also, the substance or combination of substances may include a liquid or gel disposed on the surface of substrate 210, and/or impregnated into the material of substrate 210, that may act to capture particles 213 and improve the efficiency of processing and/or improve the biological viability of particles 213. In some case the perimeter of substrate 210 may be free of the combination of substances in order to reduce/improve the degree of stiction between substrate 210 and receptacle 220 (e.g. promote slidability of substrate 210). Also, in the same or an alternative example, the perimeter of substrate 210 may include another substance that reduces the stiction where the substance may include a coating and/or be impregnated to some depth of the material of substrate 210 (e.g. a liquid, gel, plastic, etc.).

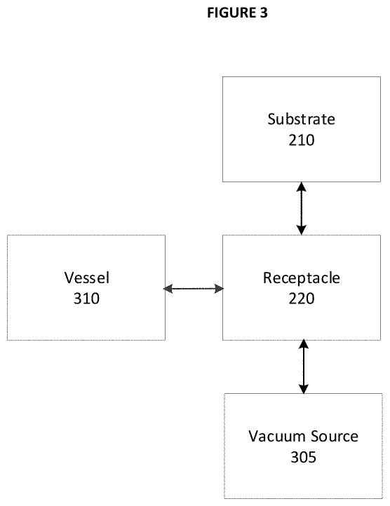

FIG. 2 also illustrates receptacle 220 which holds substrate 210 in an appropriate position relative to slit 208 for efficient capture of particles 213. FIG. 3, provides an illustrative example of the relationship between an embodiment of receptacle 220, substrate 210, vacuum source 305, and vessel 310. In the described embodiments, vacuum source 305 may include any type of source that produces a pressure differential such as a mechanical or pneumatic device (e.g. syringe pump, positive displacement pump; momentum transfer pump; regenerative pump, peristaltic pump, venturi pump, or other type of source that can create a pressure differential known in the art). In the presently described invention, the differential pressure may include a “low pressure” or “negative pressure” within vessel 310, with a differential pressure greater than about 50 mbar relative to the “high pressure” pressure side of chamber 510 with substrate 210. For example, the differential pressure may include the difference between the pressure of the ambient environment in which aerosol collector 120 is placed (e.g. atmospheric pressure), where the ambient pressure is present in chamber 510 with substrate 210 positioned therein. Vessel 310, via receptacle 220 and vacuum source 305, may include a negative pressure that creates enough pressure differential between chamber 510 and vessel 310 (e.g. vessel 310 includes a lower gas pressure than chamber 510) to move substrate 210 from its position in receptacle 220 into vessel 310. In the same or alternative example, the end of substrate 210 facing vessel 310 may include a dense material or coating that enhances the motive effect of the differential pressure on substrate 210. In the presently described example, the end may include a denser foam material or may be coated with a substantially solid material (e.g. plastic) that is effectively impermeable to gas flow.

Further, vessel 310 may include any type of vessel known in the art, particularly vessel used for safe specimen collection and transport. Many types of vessels are compatible with analytical techniques such as, for example, what may be referred to as an Eppendorf tube.

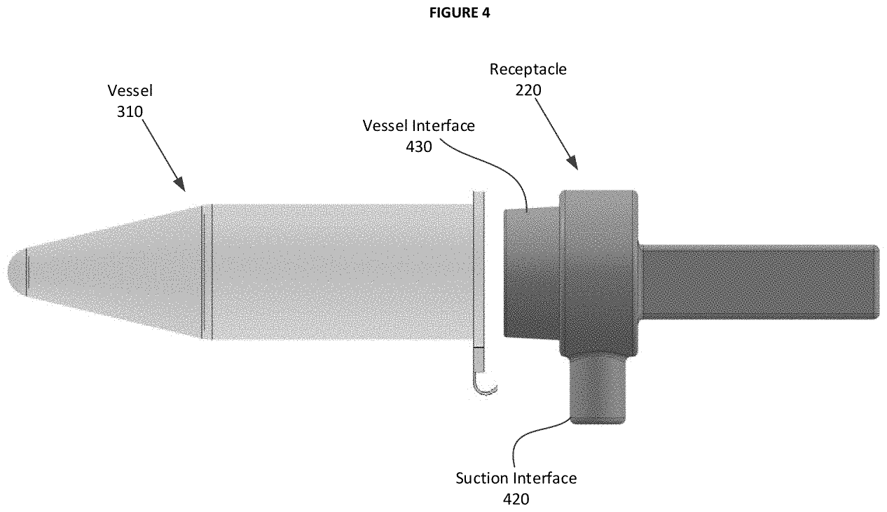

FIG. 4 provides an illustrative example of one embodiment of receptacle 220 and vessel 310, and in particular illustrating how receptacle 220 and vessel 310 operatively couple to one another. For example, receptacle 220 includes vessel interface 430 as a coupling element with vessel 310. In the example of FIG. 4, vessel interface 430 includes a tapered protrusion with a diameter at the terminal end that is smaller than an inner diameter of an open end of vessel 310. Those of ordinary skill in the art appreciate that vessel interface 430 may include an O-ring or other element to improve the seal between vessel 310 and receptacle 220, and thus the example of FIG. 4 should not be considered as limiting. When vessel interface 430 is pressed into the open end of vessel 310 a pressure tight fit is formed that may be substantially gas tight at the differential pressure range provided by vacuum source 305. In some embodiments, the pressure tight fit is reversible when a sufficient force away from receptacle 220 is applied against vessel 310, releasing vessel 310.

In the presently described example, vessel 310 may be sealable via an attached lid, separate cover, cap, etc.

FIG. 4 also illustrates suction interface 420 that may fluidically couple to vacuum source 305 via a tube, channel, or other type of fluid connection known in the art. For example, flexible tubing may fit over the outside diameter of suction interface 420 forming a pressure tight fit that is substantially gas tight at the differential pressure range provided by vacuum source 305.

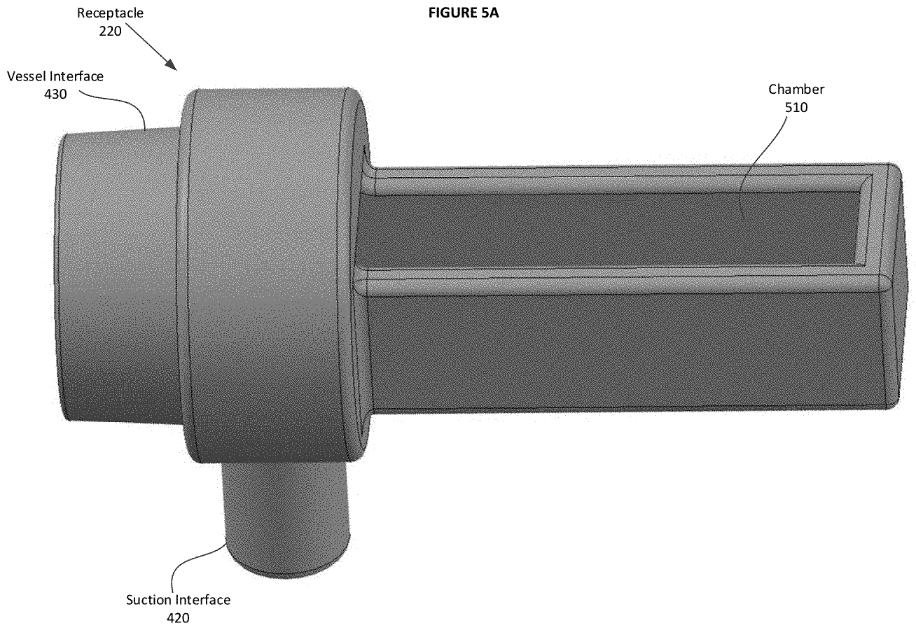

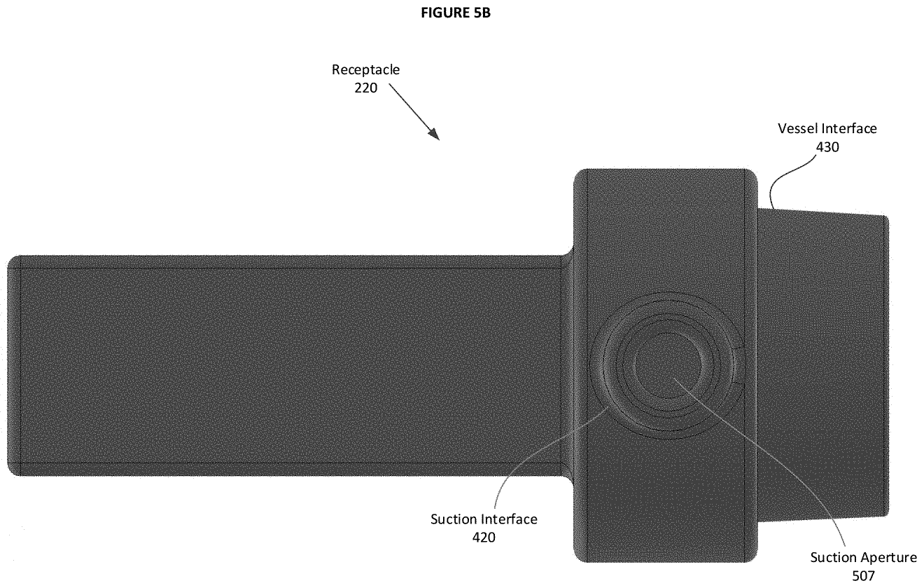

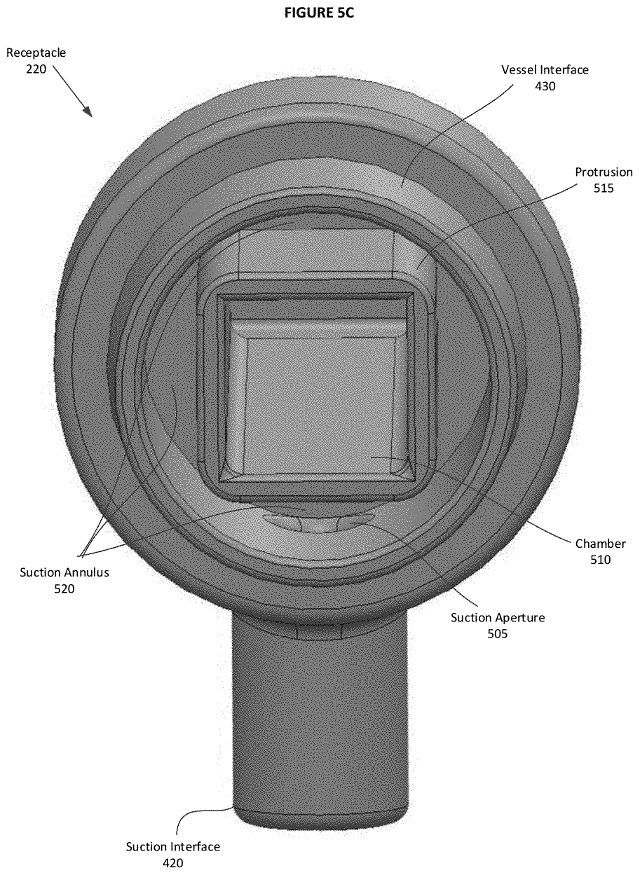

FIG. 5A provides an example of another view of receptacle 220, which shows chamber 510 constructed to receive and maintain the position of substrate 210 (e.g. limit the degree of movement of substrate 210 within chamber 510, with the exception of movement in the direction towards vessel 310. Those of skill in the art appreciate that receptacle 220 may include an elongated channel, or other configuration, between chamber 510 and vessel interface 430 in order to optimally position substrate 210 relative to impactor 150. Therefore, the example of FIG. 5A is intended to be illustrative and should not be considered as limiting.

Continuing, FIG. 5B provides a further example of a view looking at receptacle 220 from the bottom side that shows suction aperture 507 as an open end of suction interface 420.

FIG. 5C provides yet another example of a view looking at receptacle 220 from the end with vessel interface 430, and which shows protrusion 515 with an open end that provides fluid communication with chamber 510. Protrusion 515 forms suction annulus 520 that substantially surrounds protrusion 515 and helps to uniformly distribute the differential pressure. In some embodiment suction annulus may be filled with a porous material (e.g. a foam material) that is permissive to the flow of a fluid (e.g. air or other gas) but captures stray particulate. As illustrated in FIG. 5C the walls of protrusion 515 extend beyond suction aperture 505 that is in fluid communication with vacuum source 305 via suction aperture 507. For example, when receptacle 220 is operatively coupled to vessel 310, the extension of protrusion 510 causes the differential pressure from vacuum source 305 to channel through the body of vessel 310 to chamber 510 with substrate 210. The differential pressure acts on substrate 210 to cause movement of substrate 210 from chamber 510, through protrusion 515 into vessel 310 where substrate 210 is then retained, even when the differential pressure from vacuum source continues to be applied. In the presently described example motive force is applied by differential pressure created along suction annulus 520 and acts on substrate 210 through protrusion 515.

Having described various embodiments and implementations, it should be apparent to those skilled in the relevant art that the foregoing is illustrative only and not limiting, having been presented by way of example only. Many other schemes for distributing functions among the various functional elements of the illustrated embodiments are possible. The functions of any element may be carried out in various ways in alternative embodiments

What is claimed is:

1. An assembly for isolating a substrate, comprising:
a vacuum source;
a substrate;
a receptacle configured to position the substrate and to operatively couple to the vacuum source; and
a vessel configured to operatively coupled to the receptacle, wherein the substrate is configured to move from the receptacle to the vessel in response to a differential pressure applied by the vacuum source.

2. The assembly of claim 1, wherein:
the substrate is constructed of polyurethane foam.

3. The assembly of claim 1, wherein:
the substrate comprises a perimeter configured to reduce stiction.

4. The assembly of claim 1, wherein:
the substrate is configured to capture and retain a plurality of particles.

5. The assembly of claim 4, wherein:
the plurality of particles comprise biological material.

6. The assembly of claim 5, wherein:
the biological material comprises viral particles.

7. The assembly of claim 1, wherein:
the receptacle comprises a chamber that positions the substrate.

8. The assembly of claim 7, wherein:
the chamber is in fluid communication with the vessel and the vacuum source.

9. The assembly of claim 1, wherein:
the differential pressure is greater than about 50 mbar.

10. A method for isolating a substrate, comprising:
positioning a substrate in a receptacle; and
moving the substrate from the receptacle to the vessel in response to a differential pressure.
